(19) United States
(12) Patent Application Publication
(10) Pub. No.: US 2010/0210682 A1
(43) Pub. Date: Aug. 19, 2010

(54) Repeated Dosing of TRPV1 Antagonists

(75) Inventors: Connie R. Faltynek, Highland Park, IL (US); Arthur R. Gomtsyan, Vernon Hills, IL (US); Marie P. Honore, Evanston, IL (US); Michael F. Jarvis, Vernon Hills, IL (US); Shailen K. Joshi, Grayslake, IL (US); Michael E. Kort, Lake Bluff, IL (US); Philip R. Kym, Libertyville, IL (US); Torben R. Neelands, Deerfield, IL (US); Regina M. Reilly, Libertyville, IL (US); Carol S. Surowy, Sturtevant, WI (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(73) Assignee: ABBOTT LABORATORIES, Abbott Park, IL (US)

(21) Appl. No.: 12/709,125

(22) Filed: Feb. 19, 2010

Related U.S. Application Data
(60) Provisional application No. 61/153,885, filed on Feb. 19, 2009.

(51) Int. Cl. A61K 31/4725; A61K 31/416; A61K 31/444; A61K 31/421; A61P 29/00

(52) U.S. Cl. 514/310; 514/406; 514/334; 514/377

(57) ABSTRACT

Disclosed herein are methods for increasing analgesic potency of TRPV1 antagonists, said methods comprise of repeated administration of the TRPV1 antagonists in a subject. Methods for treating or preventing pain by repeated administration of a TRPV1 antagonist are also discussed.

This application claims priority to U.S. Patent Application Ser. No. 61/153,885 filed Feb. 19, 2009 which is incorporated herein by reference.

### TECHNICAL FIELD

Methods for increasing analgesic activity in a subject by repeated administration of a TRPV1 antagonist are described. In particular, methods for treating or preventing pain (e.g. inflammatory pain, bone cancer pain, osteoarthritic pain, post-operative pain) by repeated administration of a TRPV1 antagonist are disclosed.

### BACKGROUND

Pain management has become an increasing focus in medical professions in recent years as population ages. Pain may generally be classified as acute or chronic. Both differ in their etiology, pathophysiology, diagnosis, and treatment.

Morphine and other clinically employed opioids have been used for the treatment of pain. However, the analgesic benefit of morphine and related opioids can be accompanied by other undesirable side effects involving the central nervous system and gastrointestinal system including drowsiness, respiratory depression, constipation, nausea, and vomiting. There are significant adverse (respiratory depression) and dose limiting side effects. Moreover, upon repeated use, a characteristic feature of opioid drugs is the development of tolerance and physical dependence (Gilman, A. G., Goodman, L. S., Rall, T. W., and Murad, F. The Pharmacological Basis of Therapeutics. New York: MacMillan Publishing Co., 1985).

In light of the shortcomings in current approaches for treating pain, recent research have been directed towards identifying improved compositions and methods for the treatment of pain.

A wide variety of TRPV1 antagonists have been disclosed and have demonstrated their utility in the treatment of pain.

Vanilloid receptor type 1 (TRPV1) is a member of the transient receptor potential (TRP) channel superfamily. TRPV1 receptors can be activated by exogenous vanilloids (e.g. capsaicin) and by many endogenous stimuli, including heat (>43° C.), low pH, and various lipids such as anandamide that are present during inflammatory conditions. In addition to direct activation, TRPV1 activity can be enhanced by inflammatory mediators, such as prostaglandins, ATP, NGF, and bradykinin (BK) [Szallasi A, Cortright D N, Blum C A, Eld S R. The vanilloid receptor TRPV1: 10 years from channel cloning to antagonist proof-of concept. Nat Rev Drug Discov 2007; 6:357-72].

Both genetic and pharmacological approaches have shown that TRPV1 receptors play a central role in inflammatory pain transmission. Preclinical studies with multiple selective small molecule TRPV1 antagonists have demonstrated that these compounds effectively reduce inflammatory thermal hyperalgesia produced by intraplantar administration of carrageenan or complete Freund's adjuvant (CFA) in rodents. Recently, a TRPV1 antagonist with increased brain penetration has been shown to produce a broader range of analgesic activity, encompassing both mechanical and thermal endpoints in multiple rodent pain models [Cui M, Honore P, Zhong C, Gauvin D, Mikusa J, Hernandez G, et al. TRPV1 receptors in the CNS play a key role in broad-spectrum analgesia of TRPV1 antagonists. J Neurosci 2006; 26:9385-93].

TRPV1 antagonists described herein exhibit enhanced analgesic activity following repeated dosing. The analgesic potency of certain compounds increased following repeated dosing without an increase in plasma or brain drug concentrations; these effects contrast the well documented analgesic tolerance observed with opioids following repeated dosing [Honore P, Jarvis M F. Acute and chronic pain. In: Taylor J B, Triggle D J, editors. Comprehensive medicinal chemistry II, therapeutic areas I, vol. 6. Oxford: Elsevier; 2007. p. 327-49].

The observed enhanced efficacy following repeated administration of low doses of the TRPV1 antagonists may effectively reduce the required dosage to produce the desired chronic analgesic efficacy and hence result in improved therapeutic indices of the drugs.

### SUMMARY

One aspect is directed to methods for enhancing analgesic potency of TRPV1 antagonists in a subject comprising repeated administration of a TRPV1 antagonist or a pharmaceutically acceptable salt, solvate, or salt of a solvate thereof, to the subject, with or without one or more pharmaceutically acceptable carrier.

Another aspect is related to methods for treating pain comprising repeated administration of a TRPV1 antagonist or a pharmaceutically acceptable salt, solvate, or salt of a solvate thereof, to the subject, with or without one or more pharmaceutically acceptable carrier.

In certain embodiment, the TRPV1 antagonist or a pharmaceutically acceptable salt, solvate, or salt of a solvate thereof is administered with one or more pharmaceutically acceptable carrier.

In certain embodiments, the TRPV1 antagonist is repeatedly administered at a reduced dosage.

Mammalian subjects suitable for treatment by the methods described herein include, but are not limited to, those suffering from back pain (e.g. chronic low back pain), post-operative pain, injury-related pain (e.g. spinal cord injury), eye pain, inflammatory pain, bone cancer pain, osteoarthritic pain, neuropathic pain, nociceptive pain, multiple sclerosis pain, post-stroke pain, diabetic neuropathic pain, neuropathic cancer pain, trigeminal neuralgia HIV-related neuropathic pain, phantom limb pain, fibromyalgia, and migraine. In one embodiment, the subject is a human.

In certain embodiments, the TRPV1 antagonist, or a pharmaceutically acceptable salt, solvate, or salt of a solvate thereof is administered systemically, for example, via intravenous, subcutaneous, oral, topical, intranasal, sublingual, or other systemic routes. In other embodiments, it is administered centrally, for example, intrathecally. In certain embodiments, it is administered orally or intravenously. In certain embodiments, the TRPV1 antagonists or a pharmaceutically acceptable salt, solvate, or salt of a solvate thereof is administered intraperitoneally. In certain embodiments, the TRPV1 antagonists or a pharmaceutically acceptable salt, solvate, or salt of a solvate thereof is administered topically.

A therapeutic dosage amount of the TRPV1 antagonist, or a pharmaceutically acceptable salt, solvate, or salt of a solvate thereof, may be achieved by administration once daily (i.e. in a single dose), twice daily (i.e. in two separate doses), three times daily, or four times daily, over a duration of time effective to result in a diminution, and ideally elimination or ever reversal of pain. Exemplary durations of treatment include at least about 3 days, from 5 days to 1 month, from 5 days to about two weeks, from two weeks to 1 month, up to about 6 months, up to about 12 months or even longer. In one particular embodiment, treatments last from about 5 days to about 12 days. In an embodiment of the treatment method, the administration is over a duration of time effective to result in elimination of pain. In yet another embodiment, the treatments last for about 12 days. In conjunction with the previously mentioned embodiments, the TRPV1 antagonist, or a pharmaceutically acceptable salt, solvate, or salt of a solvate thereof, is administered once or twice daily.

A further aspect is related to the method for treating pain comprising repeated administration of a TRPV1 antagonist or a pharmaceutically acceptable salt, solvate, or salt of a solvate thereof, to the subject, in combination with one or more other agents effective for treating pain. Such agents include non-steroidal anti-inflammatory drugs (NSAIDs) and analgesics. In various embodiments, one or more agents are selected from the group consisting of opioid analgesics such as, but not limited to, morphine, oxycodone, or related opioids, NSAIDs such as, but not limited to, aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, meloxicam, nabumetone, naproxen, nimesulide, nitroflurbiprofen, olsalazine, oxaprozin, phenylbutazone, piroxicam, sulfasalazine, sulindac, tolmetin and zomepirac; analgesics such as, but not limited to, acetaminophen, cannabinoids, gabapentin, and memantine. In certain embodiments, exemplary agents include ibuprofen and acetaminophen. The additional agent(s) may be administered simultaneously, sequentially, or separately with the TRPV1 antagonist or pharmaceutically acceptable salt, solvate, or salt of a solvate thereof. When administered simultaneously, the additional agent(s) can be in the same or different compositions. The additional agent(s) may or may not be administered repeatedly. When administered repeatedly, the additional agent(s) may be administered repeatedly over a time course identical or different from that of the TRPV1 antagonist.

These and other objectives are described further in the following paragraphs. These objectives merely summarize certain aspects of the invention and are not intended, nor should it be construed as limiting the scope of the invention in any way.

### DETAILED DESCRIPTION

One aspect relates to methods for enhancing analgesic potency of TRPV1 antagonist,

or a pharmaceutically acceptable salt, solvate, or salt of solvate thereof, comprising administering to a subject the TRPV1 antagonist, or a pharmaceutically acceptable salt, solvate, or salt of a solvate thereof, at least once a day and repeating said administering over a period of at least 3 days, with or without one or more pharmaceutically acceptable carrier. In certain embodiments, the enhanced analgesic potency observed upon repeated administration of the TRPV1 antagonists without accumulation of the TRPV1 antagonist concentration in plasma or brain.

Another aspect relates to methods for treating pain comprising administering to a subject an effective amount of a TRPV1 antagonist or a pharmaceutically acceptable salt, solvate, or salt of a solvate thereof, at least once a day and repeating said administering over a period of at least 3 days, with or without one or more pharmaceutically acceptable carrier.

Yet another aspect relates to methods for treating pain comprising administering to a subject an effective amount of a TRPV1 antagonist or a pharmaceutically acceptable salt, solvate, or salt of a solvate thereof, at least once a day and repeating said administering over a period of at least 3 days, in combination with one or more other agents effective for treating pain.

In conjunction with any of the above or below embodiments, the TRPV1 antagonist is a compound having the structure

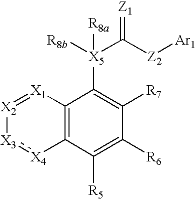

wherein

[symbol] is absent or a single bond;

X₁ is N or CR₁;

X₂ is N or CR₂;

X₃ is N, NR₃, or CR₃;

X₄ is a bond, N, or CR₄;

X₅ is N or C;

provided that at least one of X₁, X₂, X₃, and X₄ is N;

Z₁ is O, NH, or S;

Z₂ is a bond, NH, or O;

Ar₁ is selected from the group consisting of

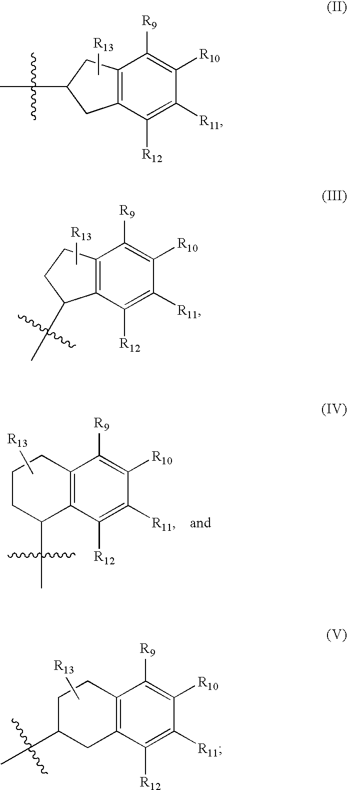

R₁, R₃, R₅, R₆, and R₇ are each independently selected from the group consisting of hydrogen, alkenyl, alkoxy, alkoxyalkoxy, alkoxyalkyl, alkoxycarbonyl, alkoxycarbonylalkyl, alkyl, alkylcarbonyl, alkylcarbonylalkyl, alkylcarbonyloxy, alkylthio, alkynyl, carboxy, carboxyalkyl, cyano, cyanoalkyl, cycloalkyl, cycloalkylalkyl, formyl, formylalkyl, haloalkoxy, haloalkyl, haloalkylthio, halogen, hydroxy, hydroxyalkyl, mercapto, mercaptoalkyl, nitro, (CF₃)₂(HO)C—, R_{B}(SO)₂R_{A}N—, R_{A}O(SO)₂—, R_{B}O(SO)₂—, Z_{A}Z_{B}N—, (Z_{A}Z_{B}N)alkyl, (Z_{A}Z_{B}N)carbonyl, (Z_{A}Z_{B}N)carbonylalkyl, and (Z_{A}Z_{B}N)sulfonyl;

R₂ and R₄ are each independently selected from the group consisting of hydrogen, alkenyl, alkoxy, alkoxyalkoxy, alkoxyalkyl, alkoxycarbonyl, alkoxycarbonylalkyl, alkyl, alkylcarbonyl, alkylcarbonylalkyl, alkylcarbonyloxy, alkylthio, alkynyl, carboxy, carboxyalkyl, cyano, cyanoalkyl, cycloalkyl, cycloalkylalkyl, formyl, formylalkyl, haloalkoxy, haloalkyl, haloalkylthio, halogen, hydroxy, hydroxyalkyl, mercapto, mercaptoalkyl, nitro, (CF₃)₂(HO)C—, R_{B}(SO)₂R_{A}N—, R_{A}O(SO)₂—, R_{B}O(SO)₂—, Z_{A}Z_{B}N—, (Z_{A}Z_{B}N)alkyl, (Z_{A}Z_{B}N)alkylcarbonyl, (Z_{A}Z_{B}N)carbonyl, (Z_{A}Z_{B}N)carbonylalkyl, (Z_{A}Z_{B}N)sulfonyl, (Z_{A}Z_{B}N)C(═NH)—, (Z_{A}Z_{B}N)C(═NCN)NH— and (Z_{A}Z_{B}N)C(═NH)NH—;

R₈ₐ is hydrogen or alkyl;

R_{8b} is absent, hydrogen, alkoxy, alkoxycarbonylalkyl, alkyl, alkylcarbonyloxy, alkylsulfonyloxy, halogen, or hydroxy;

R₉, R₁₀, R₁₁, and R₁₂ are each individually selected from the group consisting of hydrogen, alkenyl, alkoxy, alkoxyalkoxy, alkoxyalkyl, alkoxycarbonyl, alkoxycarbonylalkyl, alkyl, alkylcarbonyl, alkylcarbonylalkyl, alkylcarbonyloxy, alkylthio, alkynyl, aryl, carboxy, carboxyalkyl, cyano, cyanoalkyl, formyl, formylalkyl, haloalkoxy, haloalkyl, haloalkylthio, halogen, heteroaryl, heterocycle, hydroxy, hydroxyalkyl, mercapto, mercaptoalkyl, nitro, (CF₃)₂(HO)C—, R_{B}(SO)₂R_{A}N—, R_{A}O(SO)₂—, R_{B}O(SO)₂—, Z_{A}Z_{B}N—, (Z_{A}Z_{B}N)alkyl, (Z_{A}Z_{B}N)carbonyl, (Z_{A}Z_{B}N)carbonylalkyl, and (Z_{A}Z_{B}N)sulfonyl, wherein Z_{A} and Z_{B} are each independently hydrogen, alkyl, alkylcarbonyl, formyl, aryl, or arylalkyl, provided that at least one of R₉, R₁₀, R₁₁, or R₁₂ is other than hydrogen, or R₁₀ and R₁₁ taken together with the atoms to which they are attached form a cycloalkyl, cycloalkenyl or heterocycle ring;

R₁₃ is selected from the group consisting of hydrogen, alkyl, aryl, heteroaryl and halogen;

R_{A} is hydrogen or alkyl; and

R_{B} is alkyl, aryl, or arylalkyl;

provided that R_{8b} is absent when X₅ is N.

Examples of TRPV1 antagonists of the above structure include, but are not limited to, the examples found in US 2005/0043351, hereby incorporated by reference in its entirety.

In conjunction with any of the above or below embodiments, the TRPV1 antagonist is a compound having the above structure wherein—is absent; X₁ is CR₁; X₂ is N; X₃ is NR₃; X₄ is a bond; X₅ is N; Z₁ is O; Z₂ is NH; Ar₁ is (III); R₁, R₉, R₁₁, R₁₂, R₁₃, R₈ₐ are hydrogen; R₃ is hydrogen or alkyl; R₁₀ is alkyl; and R_{8B} is absent.

In conjunction with any of the above or below embodiments, the TRPV1 antagonist is N-[(1R)-5-tert-butyl-2,3-dihydro-1H-inden-1-yl]-N′-1H-indazol-4-ylurea (compound A).

In conjunction with any of the above or below embodiments, the TRPV1 antagonist is a compound having the structure

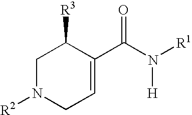

wherein

R¹ represents formula (i), (ii), (iii), or (iv)

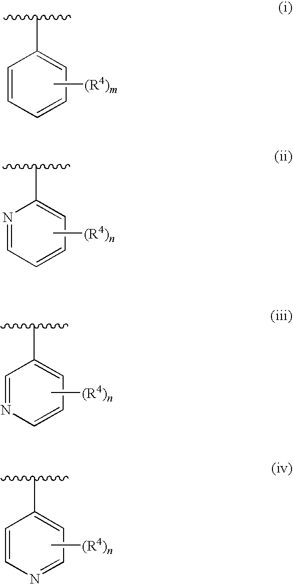

R² represents formula (v), (vi), (vii), (viii), (ix), (x), (xi), or (xii)

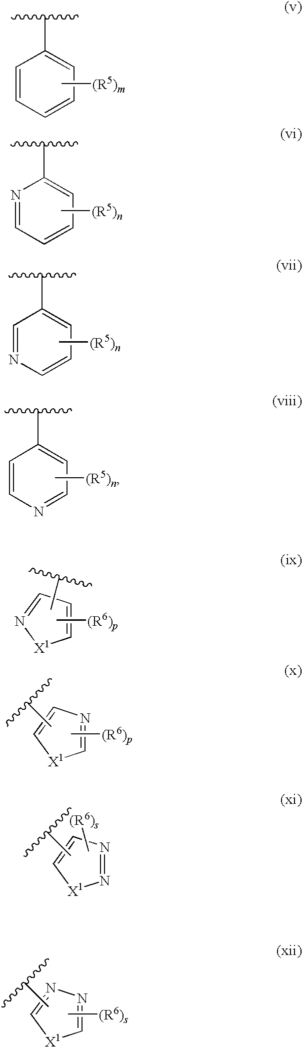

R³ is C₁₋₆ alkyl;

R⁴ represents optional substituents of R¹, and is, at each occurrence, independently alkyl, alkenyl, alkynyl, —CN, halogen, —OR^{a}, —NO₂, —N(R^{a})(R^{b}), —N(R^{b})C(O)R^{a}, —N(R^{b})S(O)₂R^{a}, —N(R^{b})C(O)OR^{a}, —N(R^{b})C(O)N(R^{a})(R^{b}), —N(R^{b})S(O)₂N(R^{a})(R^{b}), —C(O)R^{a}, —C(O)OR^{a}, —C(O)N(R^{a})(R^{b}), —S(O)₂R^{a}, —S(O)₂OR^{a}, —S(O)₂N(R^{a})(R^{b}), —(CR^{d}R^{e})_{q}—CN, haloalkyl, —(CR^{d}R^{e})_{q}—OR^{a}, —(CR^{d}R^{e})_{q}—NO₂, —(CR^{d}R^{e})_{q}—N(R^{a})(R^{b}), —(CR^{d}R^{e})_{q}—N(R^{b})C(O)R^{a}, —(CR^{d}R^{e})_{q}—N(R^{b})S(O)₂R^{a}, —(CR^{d}R^{e})_{q}—N(R^{b})C(O)OR^{a}, —(CR^{d}R^{e})_{q}—N(R^{b})C(O)N(R^{a})(R^{b}), —(CR^{d}R^{e})_{q}—N(R^{b})S(O)₂N(R^{a})(R^{b}), —(CR^{d}R^{e})_{q}—C(O)R^{a}, —(CR^{d}R^{e})_{q}—C(O)OR^{a}, —(CR^{d}R^{e})_{q}—C(O)N(R^{a})(R^{b}), —(CR^{d}R^{e})_{q}—S(O)₂R^{a}, —(CR^{d}R^{e})_{q}—S(O)₂OR^{a}, or —(CR^{d}R^{e})_{q}—S(O)₂N(R^{a})(R^{b});

R⁵ and R⁶ are optional substituents of R², and each of which at each occurrence is independently alkyl, alkenyl, alkynyl, halogen, —CN, halogen, —OR^{a}, —NO₂, —N(R^{a})(R^{b}), or haloalkyl;

R^{a} and R^{b}, at each occurrence, are each independently hydrogen, alkyl, or haloalkyl;

R^{d} and R^{e}, at each occurrence, are each independently hydrogen, alkyl, halogen, or haloalkyl;

X¹ is O or S;

m is 0, 1, 2, 3, 4, or 5;

n is 0, 1, 2, 3, or 4;

p is 0, 1, or 2;

q is 1, 2, 3, or 4; and

s is 0 or 1.

Examples of TRPV1 antagonists of the above structure include, but are not limited to, the examples found in US 2009/0062345, hereby incorporated by reference in its entirety.

In conjunction with any of the above or below embodiments, the TRPV1 antagonist is a compound having the above structure wherein R¹ is formula (I), R² is formula (vi), R³ is CH₃, R⁴ is haloalkyl or —S(O)₂R^{a}, R^{a} is alkyl or haloalkyl; R⁵ is alkyl or halogen, m is 1, and n is 1.

In conjunction with any of the above or below embodiments, the TRPV1 antagonist is (3S)-3′-chloro-3-methyl-N-[4-(trifluoromethyl)phenyl]-3,6-dihydro-2H-1,2′-bipyridine-4-carboxamide (compound B).

In conjunction with any of the above or below embodiments, the TRPV1 antagonist is a compound having the structure

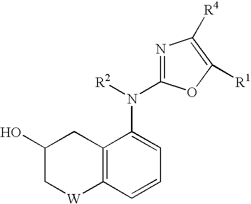

wherein

W is CH₂ or O;

R¹ is phenyl, a monocyclic heteroaryl, or a monocyclic cycloalkenyl, a monocyclic cycloalkyl, each of which is independently unsubstituted or substituted with 1, 2, 3, 4, or 5 substituents as represented by R³, wherein each R³ is independently alkyl, alkenyl, alkynyl, —NO₂, —CN, halogen, —OR^{a}, —OC(O)R^{a}, —SR^{a}, —SF₅, —S(O)R^{b}, —S(O)₂R^{b}, —S(O)₂N(R^{a})(R^{c}), —N(R^{a})(R^{c}), —N(R^{c})C(O)R^{a}, —N(R^{c})S(O)₂R^{b}, —N(R^{c})C(O)N(R^{a})(R^{c}), —N(R^{c})S(O)₂N(R^{a})(R^{c}), —C(O)R^{a}, —C(O)O(R^{a}), —C(O)N(R^{a})(R^{c}), —(CR^{e}R^{f})ₘ—CN, haloalkyl, or a monocyclic cycloalkyl that is optionally substituted with 1, 2, 3, or 4 substituents independently selected from the group consisting of alkyl, haloalkyl and halogen;

R^{a}, at each occurrence, is independently hydrogen, alkyl, or haloalkyl;

R^{b}, at each occurrence, is independently alkyl or haloalkyl;

R^{c}, at each occurrence, is independently hydrogen, alkyl, or haloalkyl;

R^{e} and R^{f} are each independently hydrogen, alkyl, or haloalkyl;

m is 1, 2, or 3;

R² is hydrogen or alkyl; and

R⁴ is methyl, ethyl, C₁-C₂ haloalkyl, or —CN.

Examples of TRPV1 antagonists of the above structure include, but are not limited to, the examples found in US 2009/0124666, hereby incorporated by reference in its entirety.

In conjunction with any of the above or below embodiments, the TRPV1 antagonist is a compound having the above structure wherein W is CH₂, R¹ is phenyl which is optionally substituted with 1, 2, or 3 substituents as represented by R³, wherein each R³ is independently alkyl, alkenyl, alkynyl, —NO₂, —CN, halogen, —OR^{a}, —SR^{a}, —SF_{S}, —S(O)R^{b}, —S(O)₂R^{b}, —S(O)₂N(R^{a})(R^{c}), —N(R^{a})(R^{c}), —C(O)R^{a}, —C(O)O(R^{a}), —C(O)N(R^{a})(R^{c}), —(CR^{e}R^{f})ₘ—CN, or haloalkyl; R^{a}, R^{c}, R^{e}, and R^{f}, at each occurrence, are each independently hydrogen or alkyl; R^{b}, at each occurrence, is independently alkyl or haloalkyl; m is 1, 2, or 3; R² is hydrogen; and R⁴ is methyl.

In conjunction with any of the above or below embodiments, the TRPV1 antagonist is (2R)-8-({4-methyl-5-[4-(trifluoromethyl)phenyl]-1,3-oxazol-2-yl}amino)-1,2,3,4-tetrahydronaphthalen-2-ol (compound C).

In conjunction with any of the above or below embodiments, the TRPV1 antagonist is a compound having the structure

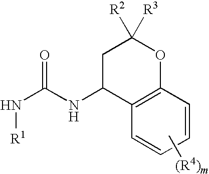

wherein

R¹ represents a group of formula (a), (b), (c), or (d)

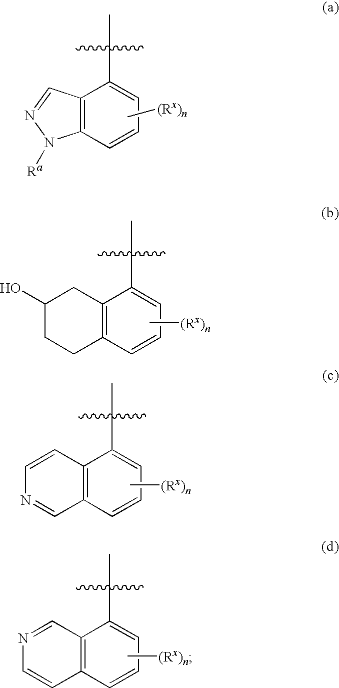

R^{x}, at each occurrence, represents optional substituent(s) on any substitutable position of the bicyclic ring selected from the group consisting of alkyl, halogen, haloalkyl, OH, O(alkyl), O(haloalkyl), NH₂, N(H)(alkyl), and N(alkyl)₂;

R^{a} is hydrogen or methyl;

R² and R³ are the same or different, and are each independently hydrogen, C₁-C₅ alkyl, or haloalkyl; or

R² and R³, together with the carbon atom to which they are attached, form a C₃-C₆ monocyclic cycloalkyl ring, optionally substituted with 1, 2, or 3 substituents selected from the group consisting of alkyl and halogen;

R⁴, at each occurrence, represents optional substituent(s) on any substitutable position of the bicyclic ring selected from the group consisting of alkyl, halogen, haloalkyl, O(alkyl), O(haloalkyl), and SCF₃; and

m and n are each independently 0, 1, 2, or 3.

Examples of TRPV1 antagonists of the above structure include, but are not limited to, the examples found in U.S. patent application Ser. No. 12/579,821 filed Oct. 15, 2009, hereby incorporated by reference in its entirety.

In conjunction with any of the above or below embodiments, the TRPV1 antagonist is a compound having the above structure wherein R¹ represents formula (b) or (c); R^{x}, at each occurrence, represents optional substituent(s) on any substitutable position of the bicyclic ring, and R^{x} is alkyl; n is 0 or 1; R² and R³ are the same or different, and are each independently C₁-C₅ alkyl or haloalkyl; R⁴, at each occurrence, represents optional substituent(s) on any substitutable position of the bicyclic ring, and is selected from the group consisting of alkyl, halogen, or haloalkyl; m is 0, 1, or 2.

In conjunction with any of the above or below embodiments, the TRPV1 antagonist is N-[(4R)-2,2-diethyl-6-fluoro-3,4-dihydro-2H-chromen-4-yl]-N′-(3-methylisoquinolin-5-yl)urea (compound D).

The following terminology will be used in accordance with the definitions described below.

It is noted that, as used in this specification and the intended claims, the singular form “a,” “an,” and “the” include plural referents unless the context clearly dictates otherwise. Thus, for example, reference to “a compound” includes a single compound as well as one or more of the same or different compounds, reference to “optional a pharmaceutically acceptable carrier” refers to a single optional pharmaceutically acceptable carrier as well as one or more pharmaceutically acceptable carriers, and the like.

The terms “effective amount” or “pharmaceutically effective amount” of a composition or agent, as provided herein, means a non-toxic but sufficient amount of the composition to provide the desired response, such as suppression of TRPV1 activation in a subject, and optionally, a corresponding therapeutic effect, such as preventing, diminishing, or eliminating pain in a subject. The exact amount required will vary from subject to subject, depending on the species, age, and general condition of the subject, the severity of the condition being treated, the particular drug or drugs employed, mode of administration, and the like. An appropriate “effective amount” in any individual case may be determined by one of ordinary skill in the art using routine experimentation.

“Treatment” or “treating” pain includes acute or chronic pain and refers to: (1) preventing pain, i.e. causing pain not to develop or occur with less intensity in a subject that may be exposed or predisposed to pain but does not yet experience or display pain, (2) inhibiting pain, i.e., arresting the development or reversing pain, or (3) relieving pain, i.e., decreasing the amount of pain experienced by the subject.

The term “subject” includes animals such as mammals, including, but not limited to, primates (e.g., humans), cows, sheep, goats, horses, dogs, cats, rabbits, rats, mice and the like. In preferred embodiments, the subject is a human.

By “reduced dosage or reduced amount” of TRPV1 antagonist is intended an amount that, when the TRPV1 antagonist is administered repeatedly at a dosage that is less than the baseline dosage, brings about a positive therapeutic response in treatment of pain, such as preventing, diminishing, or eliminating pain in a subject.

By “baseline dosage or baseline amount” of TRPV1 antagonist is intended an amount that when the TRPV1 antagonist is administered in a single dosage brings about a positive therapeutic response in treatment of pain, such as preventing, diminishing, or eliminating pain in a subject.

A reference to any one or more of the herein-described drugs is meant to encompass, where applicable, any and all enantiomers, diastereomers, mixtures of enantiomers or diastereomers, including racemates; prodrugs, pharmaceutically acceptable salts, solvates (e.g. solvates such as monohydrates, dehydrate, semi-hydrates, and the like), salts of solvates, different physical forms (e.g. crystalline solids, amorphous solids), metabolites, and the like.

The term “solvate” is used herein to describe a molecular complex comprising the active ingredient and one or more pharmaceutically acceptable solvent molecules, for example, water, ethanol, and the like.

The compounds described herein are selective and potent TRPV1 antagonists that reduce inflammatory pain as well as nociception associated with other disease-relevant preclinical models such as bone cancer pain, osteoarthritic pain, and post-operative pain. Repeated administration of the compounds at doses substantially increases the analgesic activity in multiple pain models relative to their acute effects. The enhanced antinociceptive activity following repeated dosing is in contrast with the effects observed following repeated dosing with other analgesic compounds such as opioids or non-steroidal anti-inflammatory drugs [Honore P, Jarvis M F. Acute and chronic pain. In: Taylor J B, Triggle D J, editors. Comprehensive medicinal chemistry II, therapeutic areas I, vol. 6. Oxford: Elsevier; 2007. p. 327-49].

Thus, one embodiment provides a method for treating a disorder that may be ameliorated by inhibiting vanilloid receptor subtype 1 (TRPV1) receptor in a subject in need of such treatment. The method comprises repeated administering a TRPV1 antagonist described herein or a pharmaceutically acceptable salt, solvate, or salt of a solvate thereof, with or without one or more pharmaceutically acceptable carriers, and alone, or in combination with one or more analgesics (e.g. acetaminophen, opioids), or with one or more NSAIDs, or combinations thereof.

Another embodiment provides a method for treating pain in a subject in need of such treatment. The method comprises repeated dosing of a TPRV1 antagonist described herein or a pharmaceutically acceptable salt, solvate, or salt of a solvate thereof, with or without one or more pharmaceutically acceptable carriers, and alone, or in combination with one or more analgesics (e.g. acetaminophen, opioids), or with one or more NSAIDs, or combinations thereof. For example, the method may comprises repeated dosing of a TPRV1 antagonist described herein or a pharmaceutically acceptable salt, solvate, or salt of a solvate thereof, at a reduced dosage, with or without one or more pharmaceutically acceptable carriers, and alone, or in combination with one or more analgesics (e.g. acetaminophen, opioids), or with one or more NSAIDs, or combinations thereof.

Examples of the types of pain that may be used in such treatment include, but not limited to, chronic pain, neuropathic pain, nociceptive pain, allodynia, inflammatory pain, inflammatory hyperalgesia, post herpetic neuralgia, post-operative pain, post stroke pain, neuropathies, neuralgia, diabetic neuropathy, HIV-related neuropathy, nerve injury, rheumatoid arthritic pain, osteoarthritic pain, burns, back pain, eye pain, visceral pain, cancer pain (e.g. bone cancer pain), dental pain, headache, migraine, carpal tunnel syndrome, fibromyalgia, neuritis, sciatica, pelvic hypersensitivity, pelvic pain, and menstrual pain. For example, the present methods are useful for the treatment of pain, particularly neuropathic pain, inflammatory pain, osteoarthritic pain, post-operative pain, and bone cancer pain.

TRPV1 antagonists can be used to treat pain as demonstrated by Nolano, M. et al. Pain 1999, 81, 135-145; Caterina, M. J. and Julius, D. Annu. Rev. Neurosci. 2001, 24, 487-517; Caterina, M. J. et al. Science 2000, 288, 306-313; Caterina, M. J. et al. Nature 1997, 389, 816-824.

Physiological pain is an important protective mechanism designed to warn of danger from potentially injurious stimuli from the external environment. The system operates through a specific set of primary sensory neurons and is activated by noxious stimuli via peripheral transducing mechanisms (see Millan in Prog. Neurobiol. 1999, 57, 1-164 for a review). These sensory fibers are known as nociceptors and are characteristically small-diameter axons with slow conduction velocities. Nociceptors encode the intensity, duration and quality of noxious stimulus and by virtue of their topographically organized projection to the spinal cord, the location of the stimulus. The nociceptors are found on nociceptive nerve fibers of which there are two main types, A-delta fibers (myelinated) and C fibers (non-myelinated). The activity generated by nociceptor input is transferred, after complex processing in the dorsal horn, either directly, or via brain stem relay nuclei, to the ventrobasal thalamus and then on to the cortex, where the sensation of pain is generated.

Pain may generally be classified as acute or chronic. Acute pain begins suddenly and is short-lived (usually twelve weeks or less). It is usually associated with a specific cause such as a specific injury and is often sharp and severe. It is the kind of pain that can occur after specific injuries resulting from surgery, dental work, a strain or a sprain. Acute pain does not generally result in any persistent psychological response. In contrast, chronic pain is long-term pain, typically persisting for more than three months and leading to significant psychological and emotional problems. Common examples of chronic pain are neuropathic pain (e.g. painful diabetic neuropathy, postherpetic neuralgia), carpal tunnel syndrome, back pain, headache, cancer pain, arthritic pain and chronic post-surgical pain.

When a substantial injury occurs to body tissue, via disease or trauma, the characteristics of nociceptor activation are altered and there is sensitization in the periphery, locally around the injury and centrally where the nociceptors terminate. These effects lead to a heightened sensation of pain. In acute pain, these mechanisms can be useful in promoting protective behaviors that may better enable repair processes to take place. The normal expectation would be that sensitivity returns to normal once the injury has healed. However, in many chronic pain states, the hypersensitivity far outlasts the healing process and is often due to nervous system injury. This injury often leads to abnormalities in sensory nerve fibers associated with maladaptation and aberrant activity (Woolf & Salter Science 2000, 288, 1765-1768).

Clinical pain is present when discomfort and abnormal sensitivity feature among the patient's symptoms. Patients tend to be quite heterogeneous and may present with various pain symptoms. Such symptoms include: 1) spontaneous pain which may be dull, burning, or stabbing; 2) exaggerated pain responses to noxious stimuli (hyperalgesia); and 3) pain produced by normally innocuous stimuli (allodynia: Meyer et al. Textbook of Pain, 13-44 (1994)). Although patients suffering from various forms of acute and chronic pain may have similar symptoms, the underlying mechanisms may be different and may, therefore, require different treatment strategies. Pain can also therefore be divided into a number of different subtypes according to differing pathophysiology, including nociceptive, inflammatory and neuropathic pain.

Nociceptive pain is induced by tissue injury or by intense stimuli with the potential to cause injury.

Pain afferents are activated by transduction of stimuli by nociceptors at the site of injury and activate neurons in the spinal cord at the level of their termination. This is then relayed up the spinal tracts to the brain where pain is perceived (Meyer et al. Textbook of Pain, 13-44 (1994). The activation of nociceptors activates two types of afferent nerve fibers. Myelinated A-delta fibers transmit rapidly and are responsible for sharp and stabbing pain sensations, whilst unmyelinated C fibers transmit at a slower rate and convey a dull or aching pain. Moderate to severe acute nociceptive pain is a prominent feature of pain from central nervous system trauma, strains/sprains, burns, myocardial infarction and acute pancreatitis, post-operative pain (pain following any type of surgical procedure), post-traumatic pain, renal colic, cancer pain and back pain. Cancer pain may be chronic pain such as tumor related pain (e.g. bone pain, headache, facial pain or visceral pain) or pain associated with cancer therapy (e.g. post-chemotherapy syndrome, chronic postsurgical pain syndrome or post radiation syndrome). Cancer pain may also occur in response to chemotherapy, immunotherapy, hormonal therapy or radiotherapy. Back pain may be due to herniated or ruptured intervertebral discs or abnormalities of the lumber facet joints, sacroiliac joints, paraspinal muscles or the posterior longitudinal ligament. Back pain may resolve naturally but in some patients, where it lasts over 12 weeks, it becomes a chronic condition, which can be particularly debilitating.

Neuropathic pain is currently defined as pain initiated or caused by a primary lesion or dysfunction in the nervous system. Nerve damage can be caused by trauma and disease and thus the term neuropathic pain' encompasses many disorders with diverse etiologies. These include, but are not limited to, peripheral neuropathy, diabetic neuropathy, post herpetic neuralgia, trigeminal neuralgia, back pain, cancer neuropathy, HIV neuropathy, phantom limb pain, carpal tunnel syndrome, central post-stroke pain and pain associated with chronic alcoholism, hypothyroidism, uremia, multiple sclerosis, spinal cord injury, Parkinson's disease, epilepsy and vitamin deficiency. Neuropathic pain is pathological, as it has no protective role. It is often present well after the original cause has dissipated, commonly lasting for years, significantly decreasing a patient's quality of life (Woolf and Mannion Lancet 1999, 353, 1959-1964). The symptoms of neuropathic pain are difficult to treat, as they are often heterogeneous even between patients with the same disease (Woolf and Decosterd Pain Supp. 1999, 6, S141-S147; Woolf and Mannion Lancet 1999, 353, 1959-1964). They include spontaneous pain, which can be continuous, and paroxysmal or abnormal evoked pain, such as hyperalgesia (increased sensitivity to a noxious stimulus) and allodynia (sensitivity to a normally innocuous stimulus).

The inflammatory process is a complex series of biochemical and cellular events, activated in response to tissue injury or the presence of foreign substances, which results in swelling and pain (Levine and Taiwo, Textbook of Pain, 45-56 (1994)). Arthritic pain is the most common inflammatory pain.

Rheumatoid disease is one of the commonest chronic inflammatory conditions in developed countries and rheumatoid arthritis is a common cause of disability. The exact etiology of rheumatoid arthritis is unknown, but current hypotheses suggest that both genetic and microbiological factors may be important (Grennan & Jayson, Textbook of Pain, 397-407 (1994)). It has been estimated that almost 16 million Americans have symptomatic osteoarthritis (OA) or degenerative joint disease, most of whom are over 60 years of age, and this is expected to increase to 40 million as the age of the population increases, making this a public health problem of enormous magnitude (Houge & Mersfelder Ann. Pharmacother. 2002, 36, 679-686; McCarthy et al., Textbook of Pain, 387-395 (1994)). Most patients with osteoarthritis seek medical attention because of the associated pain. Arthritis has a significant impact on psychosocial and physical function and is known to be the leading cause of disability in later life Ankylosing spondylitis is also a rheumatic disease that causes arthritis of the spine and sacroiliac joints. It varies from intermittent episodes of back pain that occur throughout life to a severe chronic disease that attacks the spine, peripheral joints and other body organs. Fernihough, J. et al. describe in Neurosci. Lett. 2005, 75-80 a potential role for TRPV1 in the manifestation of pain behavior accompanied by osteoarthritis changes in the knee.

Compounds described herein are TRPV1 antagonists and thus are useful in ameliorating acute and chronic inflammatory pain and post-operative pain as demonstrated in Honore, P. et al. J. Pharmacol. Exp. Ther. 2005, 410-421.

Another type of inflammatory pain is visceral pain, which includes pain associated with inflammatory bowel disease (IBD). Visceral pain is pain associated with the viscera, which encompass the organs of the abdominal cavity. These organs include the sex organs, spleen and part of the digestive system. Pain associated with the viscera can be divided into digestive visceral pain and non-digestive visceral pain.

Commonly encountered gastrointestinal (GI) disorders that cause pain include functional bowel disorder (FBD) and inflammatory bowel disease (IBD). These GI disorders include a wide range of disease states that are currently only moderately controlled, including, with respect to FBD, gastro-esophageal reflux, dyspepsia, irritable bowel syndrome (IBS) and functional abdominal pain syndrome (FAPS), and, in respect of IBD, Crohn's disease, ileitis and ulcerative colitis, all of which regularly produce visceral pain. Elevated TRPV1 immunoreactivity has been observed in colonic sensory nerve fibers in patients with IBD (Szallasi, A. et al. Nature Rev. 2007, 6, 357-373).

Other types of visceral pain include the pain associated with dysmenorrhea, cystitis and pancreatitis and pelvic pain.

It should be noted that some types of pain have multiple etiologies and thus can be classified in more than one area, e.g. back pain and cancer pain have both nociceptive and neuropathic components.

Other types of pain include: pain resulting from musculo-skeletal disorders, including myalgia, fibromyalgia, spondylitis, sero-negative (non-rheumatoid) arthropathies, non-articular rheumatism, dystrophinopathy, glycogenolysis, polymyositis and pyomyositis; heart and vascular pain, including pain caused by angina, myocardical infarction, mitral stenosis, pericarditis, Raynaud's phenomenon, scleredoma and skeletal muscle ischemia; head pain, such as migraine (including migraine with aura and migraine without aura), cluster headache, tension-type headache mixed headache and headache associated with vascular disorders; and orofacial pain, including dental pain, otic pain, burning mouth syndrome and temporomandibular myofascial pain. It has been shown that CGRP-receptor antagonists block the vasodilation effects of CGRP and exhibits efficacy in patients with migraine and cluster headaches. CGRP is strongly co-expressed in many TRPV1 expressing nerve fibers, it is plausible that activation of TRPV1 could partially underlie a neurogenic-mediated component of headache.

Another type of pain is ocular pain (eye pain), which includes pain associated with dry eye syndrome, increased intraocular pressure, glaucoma, accidental trauma, and surgical procedures. intraocular pressure. Activation of TRPV1 induces inflammatory cytokine release in corneal epithelium in the eye (Zhang, F. et al. J. Cell. Physiol 2007, 213, 730; Murata, Y. et al. Brain Res. 2006, 1085, 87). Retinal ganglion cell apoptosis induced by elevated hydrostatic pressure arises substantially through TRPV1, likely through the influx of extracellular Ca²⁺ (Sappington, R. M. et al. Invest. Ophth. Vis. Sci. 2009, 50, 717). TRPV1 antagonists can effectively reduce symptoms of dry eye without causing anesthesia effects on the ocular surface (US2009/0131449). Silencing of TRPV1 by administration of siRNA can be a useful therapy in the treatment of ocular pain associated with dry eye syndrome and could reduce side effects associated with medications currently used to treat patients suffering from this pathology. Investigators at Sylentis have reported data indicating that an siRNA targeting TRPV1 could be used to decrease the behavioral response of guinea pigs to ocular surface irritation (Association for Research in Vision and Opthalmology Meeting, 2008). Administration of the TRPV1 agonist capsaicin resulted in a significant increase in irritation parameters compared with saline and that topical administration of TRPV1 siRNA twice a day for three days resulted in reduced scratching and wiping movements for up to nine days in the treated eyes. The reported analgesic effect was greater than that observed using the reference standard capsazepine.

TRPV1 antagonists can be used to treat inflammatory thermal hyperalgesia as demonstrated by Davis, J. et al. Nature 2000, 405, 183-187.

TRPV1 antagonists can be used to treat bone cancer pain as demonstrated by Ghilardi J R, Rohrich H, Lindsay T H, Sevcik M A, Schwei M J, Kubota K, et al. Selective blockade of the capsaicin receptor TRPV1 attenuates bone cancer pain. J Neurosci 2005; 25:3126-31 and can block a variety of functional measures of pain including spontaneous pain associated with bone-related disease states such as bone cancer or osteoarthritis. Rich peptidergic fibers have been shown to innervate bone, the surrounding periosteum, and the joint capsule [Irie K, Fumiko H I, Ozawa H, Yajima T. Calcitonin gene-related peptide CGRP containing nerve fibers in bone tissue and their involvement in bone remodeling. Microsc Res Tech 2002; 58:85-90; Mach D B, Rogers S D, Sabino M C, Luger N M, Schwei M J, Pomonis J D, et al. Origins of skeletal pain: sensory and sympathetic innervation of the mouse femur. Neuroscience 2002; 113:155-66]. These nerves also express TRPV1 receptors [Cho W, Valtschanoff J G. Vanilloid receptor TRPV1 positive sensory afferents in the mouse ankle and knee joints. Brain Res 2008; 1219:59-65; Fernihough J, Gentry C, Malcangio M, Bevan S, Winter J. Regulation of calcitonin gene-related peptide and TRPV1 in a rat model of osteoarthritis. Neurosci Lett 2005; 388:75-80]. Furthermore, an increase in both TRPV1 receptor expression in DRG neurons and TRPV1 and CGRP receptor colocalization have been reported in the sarcoma-induced bone cancer pain model in mice [Niiyama Y, Kawamate T, Yamamoto J, Omote K, Namiki A. Bone cancer increases transient receptor potential vanilloid subfamily 1 expression within distinct subpopulations of dorsal root ganglion neurons. Neuroscience 2007; 148:560-72]. Taken together, these studies, including the present report, indicate that TRPV1 receptors play an important role in mediating bone cancer pain.

Present compounds may be administered alone, or in combination with one or more other compounds described herein, or in combination (i.e. co-administered) with one or more additional agents effective for treating pain. For example, the TRPV1 antagonist, or a pharmaceutically acceptable salt or solvate thereof, may be administered in combination with one or more analgesics (e.g. acetaminophen, or an opioid such as morphine), or with one or more nonsteroidal anti-inflammatory drug (NSAID) such as, but not limited to, aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, meloxicam, nabumetone, naproxen, nimesulide, nitroflurbiprofen, olsalazine, oxaprozin, phenylbutazone, piroxicam, sulfasalazine, sulindac, tolmetin and zomepirac; or administered with a combination of one or more analgesic (e.g. acetaminophen, opioids) and one or more NSAID. In certain embodiments, the nonsteroidal anti-inflammatory drug (NSAID) is ibuprofen. In certain embodiments, the analgesic is acetaminophen. Combination therapy includes administration of a single pharmaceutical dosage formulation containing TRPV1 antagonist and one or more additional agents, as well as administration of the TRPV1 antagonist and each additional pharmaceutical agent, in its own separate pharmaceutical dosage formulation. For example, a TRPV1 antagonist and one or more additional pharmaceutical agent(s) may be administered to the patient together, in a single oral dosage composition having a fixed ratio of each active ingredient, such as a tablet or capsule; or each agent may be administered in separate oral dosage formulations.

Where separate dosage formulations are used, the TRPV1 antagonists and one or more additional agents may be administered at essentially the same time (e.g., concurrently) or at separately staggered times (e.g., sequentially).

Actual dosage levels of active ingredients in the pharmaceutical compositions can be varied so as to obtain an amount of the active compound(s) that is effective to achieve the desired therapeutic response for a particular patient, compositions and mode of administration. The selected dosage level will depend upon the activity of the particular compound, the route of administration, the severity of the condition being treated and the condition and prior medical history of the patient being treated. However, it is within the skill of the art to start doses of the compound at levels lower than required to achieve the desired therapeutic effect and to gradually increase the dosage until the desired effect is achieved.

When used in the above or other treatments, TRPV1 antagonist and/or additional agent can be employed in pure form or, where such forms exist, in pharmaceutically acceptable salts thereof. The TRPV1 antagonist and/or additional agent can also be administered as a pharmaceutical composition comprising the compounds of interest in combination with one or more pharmaceutically acceptable carriers. The total daily usage of the compounds and compositions will be decided by the attending physician within the scope of sound medical judgment. The specific dose level necessary to achieve a positive therapeutic response in treatment of pain, such as preventing, diminishing, or eliminating pain in a subject will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well-known in the medical arts. For example, it is well within the skill of the art to start doses of the compound at levels lower than required to achieve the desired therapeutic effect and to gradually increase the dosage until the desired effect is achieved.

The total daily dose of the compounds administered to a human or lower animal range from about 0.10 μg/kg body weight to about 25 mg/kg body weight. More preferable doses can be in the range of from about 0.10 μg/kg body weight to about 1 mg/kg body weight. If desired, the effective daily dose can be divided into multiple doses for purposes of administration. Consequently, single dose compositions may contain such amounts or submultiples thereof to make up the daily dose.

Described herein are also pharmaceutical compositions comprising of a TRPV1 antagonist, or pharmaceutically acceptable salt, solvate, or salt or a solvate thereof, formulated together with one or more pharmaceutically acceptable carriers. The pharmaceutical compositions can be formulated for oral administration in solid or liquid form, for parenteral injection or for rectal administration.

The term “pharmaceutically acceptable carrier” as used herein, means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; cocoa butter and suppository waxes; oils such as peanut oil, cottonseed oil, safflower oil, sesame oil, olive oil, corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of one skilled in the art of formulations.

The pharmaceutical compositions can be administered to humans and other mammals orally, rectally, parenterally, intracisternally, intravaginally, intraperitoneally, topically (as by powders, ointments or drops), bucally or as an oral or nasal spray. The term “parenterally,” as used herein, refers to modes of administration, including intravenous, intramuscular, intraperitoneal, intrasternal, subcutaneous, intraarticular injection and infusion.

Pharmaceutical compositions for parenteral injection comprise pharmaceutically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propylene glycol, polyethylene glycol, glycerol, and the like, and suitable mixtures thereof), vegetable oils (such as olive oil) and injectable organic esters such as ethyl oleate, or suitable mixtures thereof. Suitable fluidity of the composition may be maintained, for example, by the use of a coating such as lecithin, by the maintenance of the required particle size in the case of dispersions, and by the use of surfactants.

These compositions can also contain adjuvants such as preservative agents, wetting agents, emulsifying agents, and dispersing agents. Prevention of the action of microorganisms can be ensured by various antibacterial and antifungal agents, for example, parabens, chlorobutanol, phenol, sorbic acid, and the like. It also can be desirable to include isotonic agents, for example, sugars, sodium chloride and the like. Prolonged absorption of the injectable pharmaceutical form can be brought about by the use of agents delaying absorption, for example, aluminum monostearate and gelatin.

In some cases, in order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug can depend upon its rate of dissolution, which, in turn, may depend upon crystal size and crystalline form. Alternatively, a parenterally administered drug form can be administered by dissolving or suspending the drug in an oil vehicle.

Suspensions, in addition to the active compounds, can contain suspending agents, for example, ethoxylated isostearyl alcohols, polyoxyethylene sorbitol and sorbitan esters, microcrystalline cellulose, aluminum metahydroxide, bentonite, agar-agar, tragacanth, and mixtures thereof.

If desired, and for more effective distribution, the compounds can be incorporated into slow-release or targeted-delivery systems such as polymer matrices, liposomes, and microspheres. They may be sterilized, for example, by filtration through a bacteria-retaining filter or by incorporation of sterilizing agents in the form of sterile solid compositions, which may be dissolved in sterile water or some other sterile injectable medium immediately before use.

Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides) Depot injectable formulations also are prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Injectable preparations, for example, sterile injectable aqueous or oleaginous suspensions can be formulated according to the known art using suitable dispersing or wetting agents and suspending agents. The sterile injectable preparation also can be a sterile injectable solution, suspension or emulsion in a nontoxic, parenterally acceptable diluent or solvent such as a solution in 1,3-butanediol. Among the acceptable vehicles and solvents that can be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Solid dosage forms for oral administration include capsules, tablets, pills, powders, and granules. In such solid dosage forms, one or more compounds is mixed with at least one inert pharmaceutically acceptable carrier such as sodium citrate or dicalcium phosphate and/or a) fillers or extenders such as starches, lactose, sucrose, glucose, mannitol, and salicylic acid; b) binders such as carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using lactose or milk sugar as well as high molecular weight polyethylene glycols.

The solid dosage forms of tablets, dragees, capsules, pills, and granules can be prepared with coatings and shells such as enteric coatings and other coatings well-known in the pharmaceutical formulating art. They can optionally contain opacifying agents and can also be of a composition that they release the active ingredient(s) only, or preferentially, in a certain part of the intestinal tract in a delayed manner. Examples of materials useful for delaying release of the active agent can include polymeric substances and waxes.

Compositions for rectal or vaginal administration are preferably suppositories which can be prepared by mixing the compounds with suitable non-irritating carriers such as cocoa butter, polyethylene glycol or a suppository wax which are solid at ambient temperature but liquid at body temperature and therefore melt in the rectum or vaginal cavity and release the active compound.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents.

Dosage forms for topical or transdermal administration include ointments, pastes, creams, lotions, gels, powders, solutions, sprays, inhalants or patches. A desired compound of the invention is admixed under sterile conditions with a pharmaceutically acceptable carrier and any needed preservatives or buffers as may be required. Ophthalmic formulation, eardrops, eye ointments, powders and solutions are also contemplated as being within the scope of this invention.

The ointments, pastes, creams and gels may contain, in addition to an active compound of this invention, animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of interest, lactose, talc, silicic acid, aluminum hydroxide, calcium silicates and polyamide powder, or mixtures of these substances. Sprays can additionally contain customary propellants such as chlorofluorohydrocarbons.

The active ingredients can also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes may be used. The present compositions in liposome form may contain, in addition to the compounds of interest, stabilizers, preservatives, and the like. The preferred lipids are the natural and synthetic phospholipids and phosphatidylcholines (lecithins) used separately or together.

Methods to form liposomes are known in the art. See, for example, Prescott, Ed., Methods in Cell Biology, Volume XIV, Academic Press, New York, N.Y., p 33 et seq (1976).

Dosage forms for topical administration include powders, sprays, ointments and inhalants. The active compound is mixed under sterile conditions with a pharmaceutically acceptable carrier and any needed preservatives, buffers or propellants. Ophthalmic formulations, eye ointments, powders and solutions are also contemplated as being within the scope of this invention. Aqueous liquid compositions of the invention also are particularly useful.

The active ingredients can be used in the form of pharmaceutically acceptable salts derived from inorganic or organic acids. The term “pharmaceutically acceptable salts” as used herein, include salts and zwitterions of the compounds which are, within the scope of sound medical judgment, suitable for use in contact with the tissues of humans and lower animals without undue toxicity, irritation, allergic response, and the like, are commensurate with a reasonable benefit/risk ratio, and are effective for their intended use.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well-known in the art. The salts can be prepared in situ during the final isolation and purification of the compounds or separately by mixing together solutions of the compounds of invention and a suitable acid or base. The salt may precipitate from the solution and be collected by filtration or may be recovered by evaporation of the solvent. The degree of ionization in the salt may vary from completely ionized to almost non-ionized.

Suitable acid addition salts are formed from acids which form non-toxic salts. Representative acid addition salts include, but are not limited to acetate, adipate, alginate, citrate, aspartate, benzoate, benzenesulfonate, bisulfate, bicarbonate, butyrate, camphorate, camphorsulfonate, carbonate, citrate, digluconate, glycerophosphate, hemisulfate, heptanoate, hexanoate, formate, fumarate, gluconate, glucuronate, glutamate, hydrochloride, hydrobromide, hydroiodide, 2-hydroxyethansulfonate (isethionate), lactate, maleate, malate, malonate, methanesulfonate, nicotinate, 2-naphthalenesulfonate, nicotinate, nitrate, orotate, oxalate, palmitate, pamoate, pectinate, persulfate, 3-phenylpropionate, picrate, pivalate, propionate, saccharate, stearate, succinate, sulfate, tartrate, thiocyanate, phosphate, hydrogenphosphate, dihydrogen phosphate, p-toluenesulfonate, trifluoroacetate, and undecanoate.

Also, the basic nitrogen-containing groups can be quaternized with such agents as lower alkyl halides such as methyl, ethyl, propyl, and butyl chlorides, bromides and iodides; dialkyl sulfates such as dimethyl, diethyl, dibutyl and diamyl sulfates; long chain halides such as decyl, lauryl, myristyl and stearyl chlorides, bromides and iodides; arylalkyl halides such as benzyl and phenethyl bromides and others. Water or oil-soluble or dispersible products are thereby obtained.

Basic addition salts can be prepared in situ during the final isolation and purification of compounds by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium, zinc, and aluminum salts, and the like, and nontoxic quaternary ammonia and amine cations including ammonium, tetramethylammonium, tetraethylammonium, methylamine, dimethylamine, trimethylamine, triethylamine, diethylamine, and ethylamine. Other representative organic amines useful for the formation of base addition salts include ethylenediamine, ethanolamine, diethanolamine, piperidine, and piperazine.

The term “pharmaceutically acceptable prodrug” or “prodrug” as used herein, represents those prodrugs of the active ingredients which are, within the scope of sound medical judgment, suitable for use in contact with the tissues of humans and animals without undue toxicity, irritation, allergic response, and the like, commensurate with a reasonable benefit/risk ratio, and effective for their intended use. Prodrugs can be rapidly transformed in vivo to a parent compound, for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

### In Vivo Methodology

### A. Animals

Male Sprague-Dawley rats (Charles River, Wilmington, Mass.) weighing 200-300 grams were utilized in most experiments. Animals were group housed in AAALAC approved facilities at Abbott Laboratories in a temperature-regulated environment with lights on between 0700 and 2000 h. Food and water were available ad libitum except during testing. All animal handling and experimental protocols were approved by an Institutional Animal Care and Use Committee (IACUC). All experiments were performed during the light cycle. The bone cancer pain experiments were performed on adult male C3H/HeJ mice (Jackson Laboratories, Bar Harbor, Me.), approximately 7-8 weeks old, weighing 25-30 g at the time of tumor cell injection. The mice were housed in accordance with National Institutes of Health Guidelines and kept in a vivarium maintained at 22° C. with a 12 h alternating light-dark cycle. They were given food and water ad libitum except during testing. All procedures were approved by the Institutional Animal Care and Use Committee at the University of Minnesota. All procedures also adhered to the guidelines of the Committee for Research and Ethical Issues of IASP published in PAIN, 16 (1983) 109-110.

### B. Evaluation of Analgesia Effect Assays

Compounds A, B, C, and D were evaluated in in-vivo models to assess inflammatory, osteoarthritic, post-operative, and bone cancer pain [Jarvis M F, Honore P, Shieh C C, Chapman M, Joshi S, Zhang X F, et al. A-803467, a potent and selective Nav1.8 sodium channel blocker, attenuates neuropathic and inflammatory pain in the rat. Proc Natl Acad Sci USA 2007; 104:8520-5; Schwei M J, Honore P, Rogers S D, Salak-Johnson J L, Finke M P, Ramnaraine M L, et al. Neurochemical and cellular reorganization of the spinal cord in a murine model of bone cancer pain. J Neurosci 1999; 19:10886-9]. The specific methodologies for these models are described below. Unless otherwise noted, all experimental and control groups contained at least six animals per group and data are expressed as mean±SEM. Data analysis was conducted using analysis of variance and appropriate post-hoc comparisons (p<0.05). ED50 values were estimated using least squares linear regression.

(i) Thermal Testing in Rats

The response to acute thermal stimulation was determined using a commercially available paw thermal stimulator (UARDG, University of California, San Diego, Calif.). Rats were placed individually in Plexiglass cubicles mounted on a glass surface maintained at 30° C., and allowed a 30 min habituation period. A thermal stimulus, in the form of radiant heat emitted from a focused projection bulb, was then applied to the plantar surface of each hind paw. In each test session, each rat was tested in three sequential trials at approximately 5 min intervals. Paw withdrawal latencies (PWLs) were calculated as the mean of the two shortest latencies. An assay cut-off was set at 20.5 s.

(ii) Mechanical Testing in Rats

Animals were tested for mechanical allodynia using calibrated von Frey filaments (Stoelting, Wood Dale, Ill.). Briefly, rats were placed into individual Plexiglas containers and allowed to acclimate for 15-20 minutes before testing. Paw withdrawal threshold was determined by increasing and decreasing stimulus intensity and estimated using a Dixon non-parametric test. Only rats with threshold scores 64.5 g were considered allodynic and utilized in compound testing experiments.

(iii) Capsaicin-Induced Acute Pain in Rats

Rats were placed in individual observation cages. Following an acclimation period of 30 min, the test compound was administered. Two hours later, 2.5 μg of capsaicin in a 10 μL solution of 10% ethanol/90% hydroxypropyl-b-cyclodextrin was injected subcutaneously into the dorsal aspect of the right hind paw. The observation cage was then suspended above mirrors in order to facilitate observation of the rat. Rats were observed for a continuous period of 5 minutes. The number of flinching behaviors of the injured paw was recorded during the 5 minutes observation period.

(iv) Inflammatory Pain Model in Rats

Unilateral inflammation was induced by injecting 150 μL of a 50% solution of complete Freund's adjuvant (CFA) (Sigma, St. Louis, Mo.) in physiological saline into the plantar surface of the right hind paw of the rat. The hyperalgesia to thermal stimulation was determined 2 days following CFA injection using the same apparatus as described above for the noxious acute thermal assay.

(v) Post-Operative Pain Model in Rats

As described by Brennan et al. [Brennan T J, Vandermeulen E P, Gebhart G F. Characterization of a rat model of incisional pain. Pain 1996; 64:493-501], a 1 cm longitudinal incision was made through the skin and fascia of the plantar aspect of the foot, starting 0.5 cm from the proximal edge of the heel and extending toward the toes. The plantaris muscle was elevated and incised longitudinally with origin and insertion of the muscle remaining intact. The skin was then closed with two 5-0 nylon mattress sutures. After surgery, the animals were allowed to recover and housed individually with soft bedding. Animals were tested for mechanical allodynia using von Frey hairs and for thermal hyperalgesia as described above.

(vi) Osteoarthritic Pain Model in Rats

Unilateral knee joint osteoarthritis was induced in the rats by a single intraarticular (i.a.) injection of sodium monoiodoacetate (MIA) (Sigma, St. Louis, Mo.) (3 mg in 0.05 mL sterile isotonic saline) into the joint cavity using a 26 G needle under light (2-4%) halothane (Halocarbon Laboratories, River Edge, N.J.) anesthesia [Bove S E, Calcaterra S L, Brooker R M, Huber C M, Guzman R E, Juneau P L, et al. Weight bearing as a measure of disease progression and efficacy of anti-inflammatory compounds in a model of monosodium iodoacetate-induced osteoarthritis. Osteoarthritis Cartilage 2003; 11:821-30]. Following injection, the animals were allowed to recover from the effects of anesthesia (usually 5-10 min) before returning them to their home cages.

MIA-induced nociception can be assessed by measuring differences in weight bearing (WBD) or decrease in grip force (GF) several days to weeks following MIA injection [Bove S E, Calcaterra S L, Brooker R M, Huber C M, Guzman R E, Juneau P L, et al. Weight bearing as a measure of disease progression and efficacy of anti-inflammatory compounds in a model of monosodium iodoacetate-induced osteoarthritis. Osteoarthritis Cartilage 2003; 11:821-30; Fernihough J, Gentry C, Malcangio M, Fox A, Rediske J, Pellas T, et al. Pain related behaviour in two models of osteoarthritis in the rat knee. Pain 2004; 112:83-93].

Differences in weight bearing on the injured vs uninjured hind limb were assessed by placing the animals in an Incapacitance Tester (Linton, Stoelting, Wood Dale, Ill.). The animals were restrained in a clear Plexiglass chamber (6″×3.5″×3.7″) and their hind limbs were positioned over two force plates (2″×1.5″ each) placed side by side to measure the weight borne on each hind limb. The animals were allowed to acclimate for approximately 30 s before weight-bearing readings (measured in grams) were recorded. Bilateral hind limb weight bearing, consisting of three consecutive trials (3 s/trial) was recorded for each animal and then averaged to give a mean weight-bearing score for both injured and uninjured hind limbs. Hind limb weight-bearing difference was calculated as a difference in weight bearing between the uninjured and injured limbs. Animals were tested approximately 4 and/or 21 days after MIA injection.

In addition to weight-bearing differences, grip strength was also assessed in osteoarthritic rats approximately 21 days after MIA injection. Measurements of peak hind limb grip force were conducted by recording the maximum compressive force exerted on the hind limb strain gauge setup, in a commercially available grip force measurement system (Columbus Instruments, Columbus, Ohio). During testing, each rat was gently restrained by grasping around its rib cage and then allowed to grasp the wire mesh frame (10-12 cm²) attached to the strain gauge. The experimenter then moved the animal in a rostral-to-caudal direction until the grip was broken. Each rat was sequentially tested twice at an approximately 2 min interval to obtain a raw mean grip force (CFₘₐₓ). These raw mean grip force data were in turn converted to a maximum hind limb compressive force (CFₘₐₓ) (gram force)/kg body weight for each animal. A group of age-matched naïve animals was added to each experiment and the data obtained from the different dose groups for the compound being tested were compared to the naïve group.

(vii). Bone Cancer Pain Model in Mice

An arthrotomy was performed following induction of general anesthesia with sodium pentobarbital (50 mg/kg, intraperitoneal (i.p.)). A needle was inserted into the intramedullary canal to create a pathway for the sarcoma cells. A depression was then made using a pneumatic dental high-speed hand-piece. Mice were injected with Hanks-buffered saline (HBSS) (20 μL, Sigma, St. Louis, Mo.) or HBSS containing the 2472 sarcoma line (ATCC, Rockville, Md., USA). The injection site was sealed with a dental amalgam plug to confine the cells within the intramedullary canal followed by irrigation with sterile water. Finally, incision closure was achieved with a wound clip. Clips were removed at day 5 so as not to interfere with behavioral testing.

A variety of behavioral measurements was used to assess the extent of bone cancer pain as described previously [Schwei M J, Honore P, Rogers S D, Salak-Johnson J L, Finke M P, Ramnaraine M L, et al. Neurochemical and cellular reorganization of the spinal cord in a murine model of bone cancer pain. J Neurosci 1999; 19:10886-97]. All mice were tested and values obtained for behavioral responses before compound or vehicle administration, and then retested 1 hour following oral dosing. Percent effects were calculated as follows: (1−((Compound Value−Sham Value)/(Vehicle Value−Sham Value)))×100. The number of spontaneous guarding behaviors, representative of nociceptive behavior, was recorded during a 2 min observation period. Guarding was defined as the amount of time an animal held the hind paw aloft while not ambulatory. Mechanical allodynia at the knee joint was evaluated by normally non-noxious palpation of the distal femur every second for 2 min. Following the 2 min palpation, the mice were placed in the observation box and their palpation-induced guarding behavior was measured for an additional 2 min. Normal limb use during spontaneous ambulation in an open field was scored on a scale of 5-0: (5) normal use, (4) some limping, but not pronounced, (3) pronounced limp, (2) limp and guarding behavior, (1) partial non-use of limb, and (0) complete lack of limb use.

(viii) Compounds

Compound A was dissolved in 100% polyethylene glycol (PEG 400) for oral administration in a 2 mL/kg injection volume. Compounds B, C, and D were dissolved in 10% ethanol, 20% Tween 80 and 70% polyethylene glycol-400 for oral administration in a 2 mL/kg injection volume. All compounds were dosed orally (p.o.) 60 minutes before behavioral testing.

(viii) Results

In Vivo Effects of Compound A Following Acute Dosing

Consistent with its potent ability to block TRPV1 receptor activation by capsaicin in vitro, Compound A dose-dependently prevented capsaicin-induced flinching behaviors in the rat, demonstrating that Compound A behaves as a TRPV1 receptor antagonist in vivo. In addition, Compound A dose-dependently reversed (ED₅₀=10 μmol/kg, p.o.) inflammatory thermal hyperalgesia induced by intraplantar administration of CFA

Compound A dose-dependently reversed (ED₅₀=30 μmol/kg, p.o.) the difference in weight bearing observed 4 days following MIA injection and dose-dependently reversed (ED₅₀ of 10 μmol/kg, p.o.) the decrease in grip force observed 20 days following MIA injection.

In a rodent model of post-operative pain, Compound A effectively (ED₅₀ of 40 μmol/kg, p.o.) reversed thermal hyperalgesia 24 h post-injury.

Acute dosing of Compound A fifteen days following tumor cells inoculation reversed multiple behavioral measures of pain (e.g. spontaneous pain, ambulation-evoked pain, and palpation-evoked pain) in a mouse model of bone cancer pain. Compound A (30 μmol/kg, p.o.) produced a significant 70±1% reversal of spontaneous guarding and a significant 85±15% reversal of the pain-induced decrease in ambulation. In addition to being efficacious in reducing behaviors associated with spontaneous pain, Compound A also significantly reversed evoked pain induced by palpation of the cancerous limb (65±4% effects at 30 μmol/kg, p.o.).

In Vivo Effects of Compounds A, B, C, and D Following Repeated Dosing

In the osteoarthritic pain model, animals were dosed twice-daily for 12 days using a dose that was non-efficacious following a single administration (e.g. 3 μmol/kg p.o. for Compound A). The animals were tested 1 hour after acute dosing (p.o.) and on day 12, 1 hour after oral dosing with either vehicle or the test compound. MIA injection into the knee induced a decrease in hind limb grip force that was still observed following repeated dosing with vehicle twice-daily for 12 days demonstrating that the pain had not resolved naturally during the course of chronic dosing.

Table 1 shows the effects on grip force model following acute and repeated dosing of test compounds on MIA-induced osteoarthritic rats. The analgesic responses were shown as % effect relative to the healthy, untreated rats (naïve group).

TABLE 1

| | | | % Efficacy at | |
|---|---|---|---|---|
| | Acute ED50 | | Reduced Dose | |
| | (μmol/kg; | Dose | | Chronic |
| Compound | 95% Cl) | (μmol/kg) | Acute | (BID, 12 days) |
| A | 8 | 3 μmol/kg | 20 | 62 |
| | (6.5-10) | | | |
| B | 17 | 12 μmol/kg | 30 | 80 |
| | (10-25) | | | |
| C | 14 | 5 μmol/kg | 15 | 38 |
| | (11-20) | | | |
| C | 14 | 15 μmol/kg | 22 | 83 |
| | (11-20) | | | |
| D | 26 | 10 μmol/kg | 28 | 62 |
| | (16-38) | | | |

As shown in Table 1, the analgesic effects of Compounds A, B, C, and D were significantly increased following repeated dosing. The enhanced analgesic activity of the compounds following repeated dosing was not associated with accumulation of the compound in the plasma (Table 2). These effects were also not due to the presence of active metabolites.

TABLE 2

| | |
|---|---|
| Acute ED50 | |
| (μmol/kg; | Plasma Levels (ng/mL) |

| Compound | 95% Cl) | Acute EC₅₀ | Chronic* |
|---|---|---|---|
| A | 8 | 85** | 17** at 3 μmol/kg |
| | (6.5-10) | | |
| B | 17 | 915 | 760 at 12 μmol/kg |
| | (10-25) | | |
| C | 14 | 500 | 86 at 5 μmol/kg |
| | (11-20) | | |
| C | 14 | 500 | 150 at 15 μmol/kg |
| | (11-20) | | |
| D | 26 | 1000 | 217 at 10 μmol/kg |
| | (16-38) | | |

*Plasma level was measured 1 hour after last dose on 12 days of BID dosing.
**Derived from modeling of PK/PD date from acute and chronic dosing studies in osteoarthritic rats.

Repeated dosing of Compound A produced a similar increase in analgesic activity in the bone cancer pain model. A single dose of Compound A (10 μmol/kg, p.o.) produced only an 18±6% analgesic effect on spontaneous guarding. In contrast, repeated dosing with 10 μmol/kg, p.o. of compound A twice-daily for 9 days produced 45±3% reversal of spontaneous guarding. Similarly, a single dose of Compound A (10 μmol/kg, p.o.) produced only a 19±0% analgesic effect on spontaneous ambulation, whereas repeated dosing produced a 43±0% effect. Improved analgesic efficacy following repeated dosing of Compound A was also observed on evoked pain induced by palpation of the cancerous limb.

It is understood that the foregoing detailed description and accompanying examples are merely illustrative and are not to be taken as limitations upon the scope of the invention, which is defined solely by the appended claims and their equivalents. Various changes and modifications to the disclosed embodiments will be apparent to those skilled in the art. Such changes and modifications, including without limitation those relating to the chemical structures, substituents, derivatives, intermediates, syntheses, formulations and/or methods of use of the invention, may be made without departing from the spirit and scope thereof.

1. A method for increasing analgesic potency of a TRPV1 antagonist, or a pharmaceutically acceptable salt, solvate, or salt of a solvate thereof, comprising administering to a subject the TRPV1 antagonist, or a pharmaceutically acceptable salt, solvate, or salt of a solvate thereof, at least once a day and repeating said administering over a period of at least 3 days.

2. A method for treating or preventing pain in a subject comprising administering to a subject an effective amount of a TRPV1 antagonist, or a pharmaceutically acceptable salt, solvate, or salt of a solvate thereof, at least once a day and repeating said administering over a period of at least 3 days.

3. The method according to claim 2 wherein the pain to be treated or prevented is selected from the group consisting of neuropathic pain, bone cancer pain, inflammatory pain, and osteoarthritic pain.

4. The method according to claim 2 wherein the subject is human.

5. The method according to claim 2 wherein the effective amount of TRPV1 antagonist, or a pharmaceutically acceptable salt, solvate, or salt of a solvate thereof is the reduced dosage.

6. The method according to claim 2 wherein the TRPV1 antagonist, or a pharmaceutically acceptable salt, solvate, or salt of a solvate thereof is administered orally.

7. The method according to claim 2 wherein the TRPV1 antagonist, or a pharmaceutically acceptable salt, solvate, or salt of a solvate thereof is administered topically.

8. The method according to claim 2 wherein the TRPV1 antagonist, or a pharmaceutically acceptable salt, solvate, or salt of a solvate thereof is administered once, twice, three times, or four times daily.

9. The method according to claim 2 wherein the TRPV1 antagonist, or a pharmaceutically acceptable salt, solvate, or salt of a solvate thereof is administered repeatedly over a period of 5 days to 2 weeks.

10. The method according to claim 2 wherein the TRPV1 antagonist, or a pharmaceutically acceptable salt, solvate, or salt of a solvate thereof is administered repeatedly over a period of at least 12 days.

11. The method according to claim 2 wherein the TRPV1 antagonist, or a pharmaceutically acceptable salt, solvate, or salt of a solvate thereof is administered repeatedly over a period of 12 days.

12. The method according to any one of claims 1-11 wherein the TRPV1 antagonist is a compound having formula 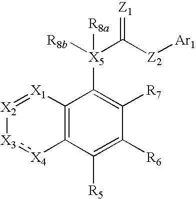
wherein [symbol]
is absent or a single bond;
X₁ is N or CR₁;
X₂ is N or CR₂;
X₃ is N, NR₃, or CR₃;
X₄ is a bond, N, or CR₄;
X₅ is N or C;
provided that at least one of X₁, X₂, X₃, and X₄ is N;
Z₁ is O, NH, or S;
Z₂ is a bond, NH, or O;
Ar₁ is selected from the group consisting of 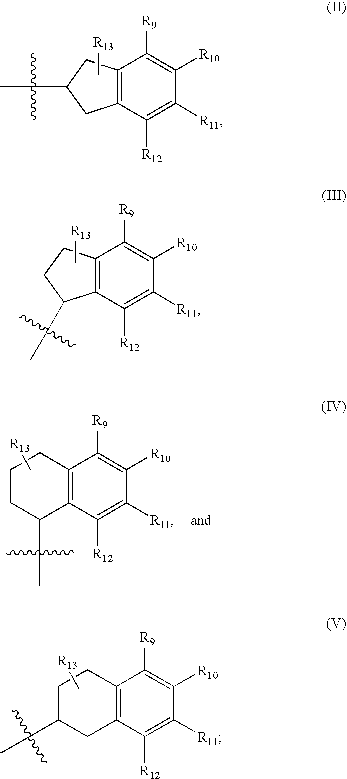
R₁, R₃, R₅, R₆, and R₇ are each independently selected from the group consisting of hydrogen, alkenyl, alkoxy, alkoxyalkoxy, alkoxyalkyl, alkoxycarbonyl, alkoxycarbonylalkyl, alkyl, alkylcarbonyl, alkylcarbonylalkyl, alkylcarbonyloxy, alkylthio, alkynyl, carboxy, carboxyalkyl, cyano, cyanoalkyl, cycloalkyl, cycloalkylalkyl, formyl, formylalkyl, haloalkoxy, haloalkyl, haloalkylthio, halogen, hydroxy, hydroxyalkyl, mercapto, mercaptoalkyl, nitro, (CF₃)₂(HO)C—, R_{B}(SO)₂R_{A}N—, R_{A}O(SO)₂—, R_{B}O(SO)₂—, Z_{A}Z_{B}N—, (Z_{A}Z_{B}N)alkyl, (Z_{A}Z_{B}N)carbonyl, (Z_{A}Z_{B}N)carbonylalkyl, and (Z_{A}Z_{B}N)sulfonyl;
R₂ and R₄ are each independently selected from the group consisting of hydrogen, alkenyl, alkoxy, alkoxyalkoxy, alkoxyalkyl, alkoxycarbonyl, alkoxycarbonylalkyl, alkyl, alkylcarbonyl, alkylcarbonylalkyl, alkylcarbonyloxy, alkylthio, alkynyl, carboxy, carboxyalkyl, cyano, cyanoalkyl, cycloalkyl, cycloalkylalkyl, formyl, formylalkyl, haloalkoxy, haloalkyl, haloalkylthio, halogen, hydroxy, hydroxyalkyl, mercapto, mercaptoalkyl, nitro, (CF₃)₂(HO)C—, R_{B}(SO)₂R_{A}N—, R_{A}O(SO)₂—, R_{B}O(SO)₂—, Z_{A}Z_{B}N—, (Z_{A}Z_{B}N)alkyl, (Z_{A}Z_{B}N)alkylcarbonyl, (Z_{A}Z_{B}N)carbonyl, (Z_{A}Z_{B}N)carbonylalkyl, (Z_{A}Z_{B}N)sulfonyl, (Z_{A}Z_{B}N)C(═NH)—, (Z_{A}Z_{B}N)C(═NCN)NH— and (Z_{A}Z_{B}N)C(═NH)NH—;
R₈ₐ is hydrogen or alkyl;
R_{8b} is absent, hydrogen, alkoxy, alkoxycarbonylalkyl, alkyl, alkylcarbonyloxy, alkylsulfonyloxy, halogen, or hydroxy;
R₉, R₁₀, R₁₁, and R₁₂ are each individually selected from the group consisting of hydrogen, alkenyl, alkoxy, alkoxyalkoxy, alkoxyalkyl, alkoxycarbonyl, alkoxycarbonylalkyl, alkyl, alkylcarbonyl, alkylcarbonylalkyl, alkylcarbonyloxy, alkylthio, alkynyl, aryl, carboxy, carboxyalkyl, cyano, cyanoalkyl, formyl, formylalkyl, haloalkoxy, haloalkyl, haloalkylthio, halogen, heteroaryl, heterocycle, hydroxy, hydroxyalkyl, mercapto, mercaptoalkyl, nitro, (CF₃)₂(HO)C—, R_{B}(SO)₂R_{A}N—, R_{A}O(SO)₂—, R_{B}O(SO)₂—, Z_{A}Z_{B}N—, (Z_{A}Z_{B}N)alkyl, (Z_{A}Z_{B}N)carbonyl, (Z_{A}Z_{B}N)carbonylalkyl, and (Z_{A}Z_{B}N)sulfonyl, wherein Z_{A} and Z_{B} are each independently hydrogen, alkyl, alkylcarbonyl, formyl, aryl, or arylalkyl, provided that at least one of R₉, R₁₀, R₁₁, or R₁₂ is other than hydrogen, or R₁₀ and R₁₁ taken together with the atoms to which they are attached form a cycloalkyl, cycloalkenyl or heterocycle ring;
R₁₃ is selected from the group consisting of hydrogen, alkyl, aryl, heteroaryl and halogen;
R_{A} is hydrogen or alkyl; and
R_{B} is alkyl, aryl, or arylalkyl;
provided that R_{8b} is absent when X₅ is N.

13. The method according to any one of claims 1-11 wherein the TRPV1 antagonist is a compound having formula 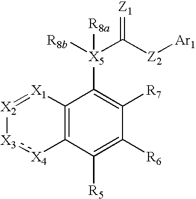
wherein [symbol]
is absent;
X₁ is CR₁;
X₂ is N;
X₃ is NR₃;
X₄ is a bond;
X₅ is N;
Z₁ is O;
Z₂ is NH;
Ar₁ is 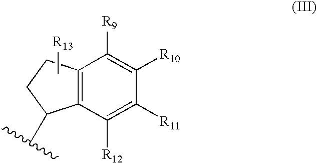
R₁, R₉, R₁₁, R₁₂, R₁₃, R₈ₐ are hydrogen;
R₃ is hydrogen or alkyl;
R₁₀ is alkyl; and
R_{8B} is absent.

14. The method according to any one of claims 1-11 wherein the TRPV1 antagonist is a compound having formula 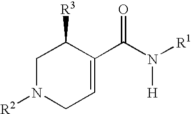
wherein
R¹ represents formula (i), (ii), (iii), or (iv) 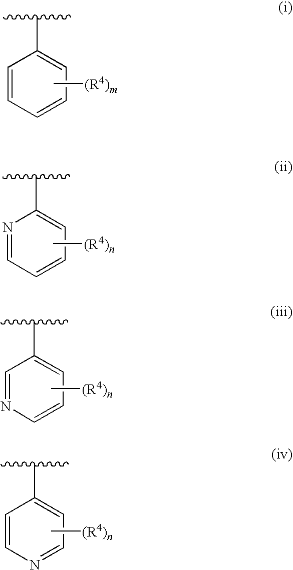
R² represents formula (v), (vi), (vii), (viii), (ix), (x), (xi), or (xii) 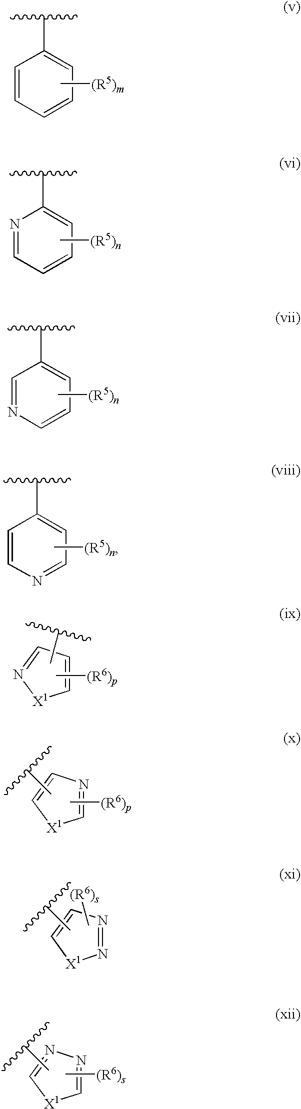
R³ is C₁₋₆ alkyl;
R⁴ represents optional substituents of R¹, and is, at each occurrence, independently alkyl, alkenyl, alkynyl, —CN, halogen, —OR^{a}, —NO₂, —N(R^{a})(R^{b}), —N(R^{b})C(O)R^{a}, —N(R^{b})S(O)₂R^{a}, —N(R^{b})C(O)OR^{a}, —N(R^{b})C(O)N(R^{a})(R^{b}), —N(R^{b})S(O)₂N(R^{a})(R^{b}), —C(O)R^{a}, —C(O)OR^{a}, —C(O)N(R^{a})(R^{b}), —S(O)₂R^{a}, —S(O)₂OR^{a}, —S(O)₂N(R^{a})(R^{b}), —(CR^{d}R^{e})_{q}—CN, haloalkyl, —(CR^{d}R^{e})_{q}—OR^{a}, —(CR^{d}R^{e})_{q}—NO₂, —(CR^{d}R^{e})_{q}—N(R^{a})(R^{b}), —(CR^{d}R^{e})_{q}—N(R^{b})C(O)R^{a}, —(CR^{d}R^{e})_{q}—N(R^{b})S(O)₂R^{a}, —(CR^{d}R^{e})_{q}—N(R^{b})C(O)OR^{a}, —(CR^{d}R^{e})_{q}—N(R^{b})C(O)N(R^{a})(R^{b}), —(CR^{d}R^{e})_{q}—N(R^{b})S(O)₂N(R^{a})(R^{b}), —(CR^{d}R^{e})_{q}—C(O)R^{a}, —(CR^{d}R^{e})_{q}—C(O)OR^{a}, —(CR^{d}R^{e})_{q}—C(O)N(R^{a})(R^{b}), —(CR^{d}R^{e})_{q}—S(O)₂R^{a}, —(CR^{d}R^{e})_{q}—S(O)₂OR^{a}, or —(CR^{d}R^{e})_{q}—S(O)₂N(R^{a})(R^{b});
R⁵ and R⁶ are optional substituents of R², and each of which at each occurrence is independently alkyl, alkenyl, alkynyl, halogen, —CN, halogen, —OR^{a}, —NO₂, —N(R^{a})(R^{b}), or haloalkyl;
R^{a} and R^{b}, at each occurrence, are each independently hydrogen, alkyl, or haloalkyl;
R^{d} and R^{e}, at each occurrence, are each independently hydrogen, alkyl, halogen, or haloalkyl;
X¹ is O or S;
m is 0, 1, 2, 3, 4, or 5;
n is 0, 1, 2, 3, or 4;
p is 0, 1, or 2;
q is 1, 2, 3, or 4; and
s is 0 or 1.

15. The method according to any one of claims 1-11 wherein the TRPV1 antagonist is a compound having formula 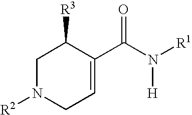
wherein
R¹ is 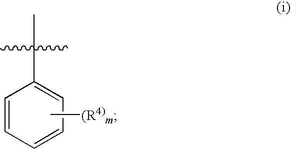
R² is 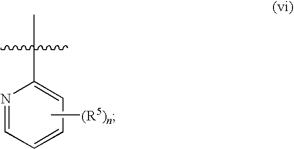
R³ is CH₃,
R⁴ is haloalkyl or —S(O)₂R^{a} wherein R^{a} is alkyl or haloalkyl;
R⁵ is alkyl or halogen;
m is 1; and
n is 1.

16. The method according to any one of claims 1-11 wherein the TRPV1 antagonist is a compound having formula 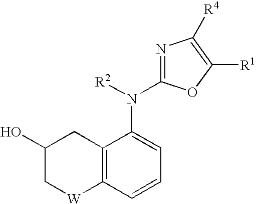
wherein
W is CH₂ or O;
R¹ is phenyl, a monocyclic heteroaryl, or a monocyclic cycloalkenyl, a monocyclic cycloalkyl, each of which is independently unsubstituted or substituted with 1, 2, 3, 4, or 5 substituents as represented by R³, wherein each R³ is independently alkyl, alkenyl, alkynyl, —NO₂, —CN, halogen, —OR^{a}, —OC(O)R^{a}, —SR^{a}, —SF_{S}, —S(O)R^{b}, —S(O)₂R^{b}, —S(O)₂N(R^{a})(R^{c}), —N(R^{a})(R^{c}), —N(R^{c})C(O)R^{a}, —N(R^{c})S(O)₂R^{b}, —N(R^{c})C(O)N(R^{a})(R^{c}), —N(R^{c})S(O)₂N(R^{a})(R^{c}), —C(O)R^{a}, —C(O)O(R^{a}), —C(O)N(R^{a})(R^{c}), —(CR^{e}R^{f})ₘ—CN, haloalkyl, or a monocyclic cycloalkyl that is optionally substituted with 1, 2, 3, or 4 substituents independently selected from the group consisting of alkyl, haloalkyl and halogen;
R^{a}, at each occurrence, is independently hydrogen, alkyl, or haloalkyl;
R^{b}, at each occurrence, is independently alkyl or haloalkyl;
R^{c}, at each occurrence, is independently hydrogen, alkyl, or haloalkyl;
R^{e} and R^{f} are each independently hydrogen, alkyl, or haloalkyl;
m is 1, 2, or 3;
R² is hydrogen or alkyl; and
R⁴ is methyl, ethyl, C₁-C₂ haloalkyl, or —CN.

17. The method according to any one of claims 1-11 wherein the TRPV1 antagonist is a compound having formula 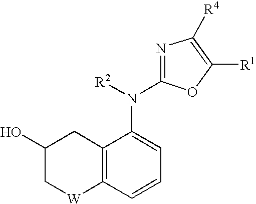
wherein
W is CH₂;
R¹ is phenyl which is optionally substituted with 1, 2, or 3 substituents as represented by R³, wherein each R³ is independently alkyl, alkenyl, alkynyl, —NO₂, —CN, halogen, —OR^{a}, —SR^{a}, —SF₅, —S(O)R^{b}, —S(O)₂R^{b}, —S(O)₂N(R^{a})(R^{c}), —N(R^{a})(R^{c}), —C(O)R^{a}, —C(O)O(R^{a}), —C(O)N(R^{a})(R^{c}), —(CR^{e}R^{f})ₘ—CN, or haloalkyl;
R^{a}, R^{c}, R^{e}, and R^{f}, at each occurrence, are each independently hydrogen or alkyl;
R^{b}, at each occurrence, is independently alkyl or haloalkyl;
m is 1, 2, or 3;
R² is hydrogen; and
R⁴ is methyl.

18. The method according to any one of claims 1-11 wherein the TRPV1 antagonist is a compound having formula 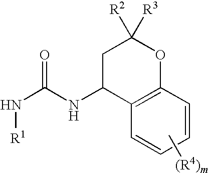
wherein
R¹ represents a group of formula (a), (b), (c), or (d) 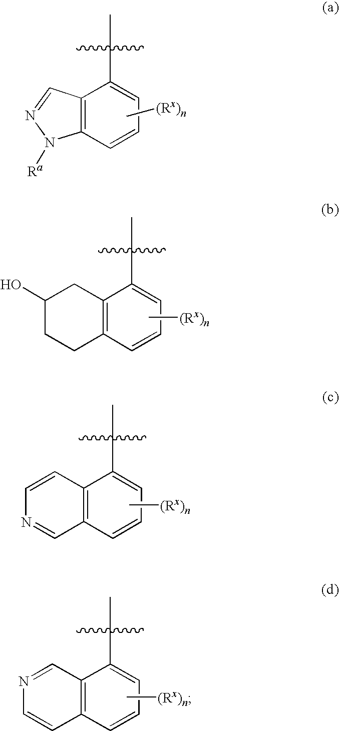
R^{x}, at each occurrence, represents optional substituent(s) on any substitutable position of the bicyclic ring selected from the group consisting of alkyl, halogen, haloalkyl, OH, O(alkyl), O(haloalkyl), NH₂, N(H)(alkyl), and N(alkyl)₂;
R^{a} is hydrogen or methyl;
R² and R³ are the same or different, and are each independently hydrogen, C₁-C₅ alkyl, or haloalkyl; or
R² and R³, together with the carbon atom to which they are attached, form a C₃-C₆ monocyclic cycloalkyl ring, optionally substituted with 1, 2, or 3 substituents selected from the group consisting of alkyl and halogen;
R⁴, at each occurrence, represents optional substituent(s) on any substitutable position of the bicyclic ring selected from the group consisting of alkyl, halogen, haloalkyl, O(alkyl), O(haloalkyl), and SCF₃; and
m and n are each independently 0, 1, 2, or 3.

19. The method according to any one of claims 1-11 wherein the TRPV1 antagonist is a compound having formula 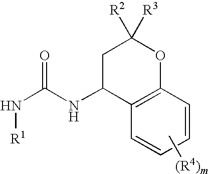
wherein
R¹ represents a group of formula (b) or (c); 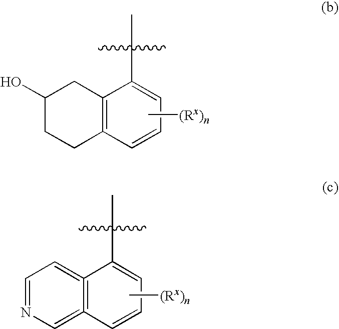
R^{x}, at each occurrence, represents optional substituent(s) on any substitutable position of the bicyclic ring, and R^{x} is alkyl;
n is 0 or 1;
R² and R³ are the same or different, and are each independently C₁-C₅ alkyl or haloalkyl;
R⁴, at each occurrence, represents optional substituent(s) on any substitutable position of the bicyclic ring, and is selected from the group consisting of alkyl, halogen, or haloalkyl; and
m is 0, 1, or 2.

20. The method according to any one of claims 1-11 wherein the TRPV1 antagonist is a compound selected from the group consisting of
N-[(1R)-5-tert-butyl-2,3-dihydro-1H-inden-1-yl]-N′-1H-indazol-4-ylurea;
(3S)-3′-chloro-3-methyl-N-[4-(trifluoromethyl)phenyl]-3,6-dihydro-2H-1,2′-bipyridine-4-carboxamide;
(2R)-8-({4-methyl-5-[4-(trifluoromethyl)phenyl]-1,3-oxazol-2-yl}amino)-1,2,3,4-tetrahydronaphthalen-2-ol; and
N-[(4R)-2,2-diethyl-6-fluoro-3,4-dihydro-2H-chromen-4-yl]-N-(3-methylisoquinolin-5-yl)urea.
